(19) United States
(12) Patent Application Publication
(10) Pub. No.: US 2010/0152565 A1
(43) Pub. Date: Jun. 17, 2010

(54) NON-INVASIVE TONOMETER

(76) Inventors: Gordon A. Thomas, Princeton, NJ (US); Tara L. Alvarez, Whippany, NJ (US); Robert Fechtner, Watchung, NJ (US); Stephanie M. Milczarski, Montclair, NJ (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/503,700

(22) Filed: Jul. 15, 2009

Related U.S. Application Data
(60) Provisional application No. 61/080,859, filed on Jul. 15, 2008.

(51) Int. Cl. A61B 3/16

(52) U.S. Cl. 600/405

(57) ABSTRACT

Tonometers are disclosed for measuring intraocular pressure (IOP) and having an ocular probe movable a predetermined distance in a linear manner by a motor against the closed eyelid of a patient's test eye, a distance sensor configured to monitor the probe position as the probe is moved against the eyelid and provide a distance measurement, a mechanism for aligning the probe with the center of the cornea underneath the closed eyelid, and a force sensor configured to measure force on the ocular probe as it is moved against the closed eyelid by the motor and provide a force measurement, wherein a value indicative of IOP of the test eye is determined from the force and distance measurements. Methods for measuring IOP using the inventive tonometers are also disclosed.

### CROSS-REFERENCE TO RELATED APPLICATIONS

This application claims the benefit of U.S. Provisional Application Ser. No. 61/080,859, filed Jul. 15, 2008, the disclosure of which is incorporated herein by reference in its entirety.

### FIELD OF THE INVENTION

This invention relates to methods and devices for measuring intraocular pressure (IOP) and, in particular, to non-invasive tonometers for self-administered IOP measurements.

### BACKGROUND

Glaucoma is a disease that affects millions of people across the globe, about 3 million Americans suffer from this disease and 12 million more are at a risk of developing the disease. It is said to be the second leading cause of blindness and is correlated with an elevated intraocular pressure (IOP). In the standard model, the rise in intraocular pressure results when there is excessive aqueous humor in the anterior chamber of the eye because of the imbalance between the quantity of fluid secreted from the ciliary body and that drained through the trabecular meshwork. Since the chamber cannot increase in size, the fluid presses against the retina walls, compressing and damaging the cells along the optic nerve, causing the cells to die which leads to loss of vision.

A number of different tonometers have been developed over the years to measure IOP. Most of the existing tonometers, however, can only be used in clinical settings by health care professionals, such as ophthalmologists or optometrists. But, the IOP is not a constant value but fluctuates throughout the day with a 24-hour periodicity of circadian rhythms and hence necessitates measurement outside typical health care professional office hours. Accordingly, there is still a need for patient-operated tonometers that are easy to use and provide reliable IOP measurements.

### SUMMARY

The instant disclosure provides tonometers that can easily be used outside the health professional's office. The tonometers of the present invention are non-invasive and measure the IOP through the eyelid, so the need for anesthesia and risk of patient infection is completely eliminated. With the tonometers of the present invention, measurement of IOP can be done within fractions of a second, which eliminates the prolonged time required to position the patient before measurements can be done.

The measurement can be done either when the patient is in a supine position or sitting. The accuracy of measurements with the disclosed tonometers is not dependent on technique or the expertise of the operator. Accordingly, tonometers of the present invention are appropriate for use in non-clinical settings such as at a patient's home or in places across the globe where an opthalmologic service is not readily available. However, the tonometers of the present invention can be used in clinical settings as well, and the invention as presently claims should not be construed as limited to self-measurement devices or methods of self-measurement.

Therefore, one aspect of the present invention provides a tonometer for measuring IOP, with an ocular probe movable in a linear manner by a motor against the closed eyelid of a user's eye to be tested; a distance sensor configured to monitor the probe's position; a mechanism for aligning the ocular probe with the center of the cornea underneath the closed eyelid; and a force sensor configured to measure force on the ocular probe as it is moved against the closed eyelid by the motor at the cornea center. The tonometer may be connected to a data acquisition unit with a processor and memory coupled to processor, in which the memory contains program code executable by the processor to cause the processor to receive data from the distance sensor and the force sensor and calculate a value for IOP from the data.

To ensure reproducibility of the test results, a mechanism for aligning the ocular probe with the center of the cornea underneath said closed eyelid is provided. In one embodiment, the mechanism includes a light source, such as a light emitting diode (LED) or a laser.

In another embodiment, the mechanism utilizes the position monitor and/or force sensor to center the probe on the cornea. Because the cornea protrudes beyond the spherical radius of the eyeball and the center of the cornea protrudes the most, the force signal and/or the displacement signal will peak when the ocular probe is precisely at the center of the cornea. Accordingly, the tonometer measures the distance between the probe and the cornea through the closed eyelid, identifies the cornea center by its protrusion from the eyeball and notifies the patient when the probe is centered.

More specifically, in order to center the ocular probe, it is placed on the eyelid of the closed eye and the patient moves the open eye around, which causes the closed eye to move as well. When the data acquisition unit concludes from the signal from the force sensor and/or position monitor that the ocular probe is centered, it will notify the patient to hold still and begin the test, for example, by emitting an audible signal.

To facilitate this mechanism for centering the probe on the cornea, the memory of the data acquisition unit may further comprise a program code to perform the following steps: detecting placement of the ocular probe against a patient's eyelid; identifying when the ocular probe is centered against the patient's cornea as inferred from the signal from the force sensor and/or position monitor; and notifying the user that the ocular probe is centered so the test can commence.

Stabilizing the tonometer itself improves the accu-racy of the results. Accordingly, in some embodiments the tonometer may be secured to the patient's head using a head-mount. Alternatively, the tonometer may be fixed to a desk.

Another aspect of the present invention provides a method for the self-measurement of intraocular pressure. The method includes the steps of:

placing a tonometer having an ocular probe in proximity to a user' test eye and observing eye, wherein the tonometer has an adjustment mechanism for aligning the probe with the center of the cornea of the test eye through a closed eyelid, and emitting a signal when the probe is aligned with the cornea center;

shutting the eyelid of the test eye and placing the ocular probe in contact with said closed eyelid;

aligning the probe with the center of the cornea by moving the observing eye around in in at least one direction selected from left, right, up and down so that the closed eye follows, until a signal is received that the ocular probe is aligned with the cornea center;

advancing the probe against the closed eyelid above the cornea center;

measuring the force required to deflect the eyelid and cornea as a function of distance that the probe has moved into the eye; and

determining the value of IOP based on the force measurement.

The instant tonometer uses compressibility measurements, rather than aplanation or indentation, to monitor IOP anomalies. The design of this device will give a measurement accuracy of about +/−2 mmHg of the IOP and with standard deviation within 0.5 mmHg for a patient with an average IOP of 16 mmHg.

Besides using the device to determine or measure IOP, the device can also be used to measure compliance of the retropulsive structure in low pressure glaucoma, to measure the correlation of the IOP and intracranial pressure, and to measure ocular hysteresis, all of which can be calculated from the flexibility or compressibility of the eye.

### BRIEF DESCRIPTION OF THE FIGURES

FIG. 1a is a schematic diagram of an exemplary embodiment of the instant tonometer.

FIG. 1b shows the embodiment of the instant tonometer shown in FIG. 1a in operation.

FIG. 2 shows an embodiment of the instant tonometer having a head-mount.

FIG. 3 shows the variance in signal when centering the ocular probe on the cornea.

FIG. 4 is a block diagram of the electronics of an embodiment of the instant tonometer.

FIG. 5 shows a typical graph of force on the probe as a function of distance the probe has moved into the eye.

FIG. 6 presents data of measurements of the compressibility of the retropulsive structure in a human subject, with a cornea shield over the cornea.

FIG. 7 presents data of measurements of the compressibility of the spring in an embodiment of the instant tonometer, by taken measurements on the wall.

FIGS. 8a and 8b present comparison of linear and non-linear fit of the data obtained with an embodiment of the instant tonometer in laboratory testing and on human subjects, respectively.

FIGS. 9a-9b illustrate the reproducibility of the results obtained with an embodiment of the instant tonometer.

FIG. 10 shows the effect of stabilizing the instant tonometer on accuracy of the results.

FIG. 11 shows the effect of centering the probe on the cornea on accuracy of the results.

FIG. 12 is another example of the effect of centering the probe on the cornea on accuracy of the results.

FIG. 13 shows the effect of repositioning of the instant tonometer during a test on accuracy of the results.

FIG. 14 presents a compliance mapping of the cornea through the eyelid.

### DETAILED DESCRIPTION

Generally, the instant tonometer comprises an ocular probe movable in a linear manner by a motor against a patient's eye; a position monitor configured to monitor the probe's position; a mechanism for centering the probe on the cornea through the closed eyelid; and a force sensor configured to measure the force against the probe as the probe is moved against the user's eye. Although the data from the force sensor and position monitor may be collected and/or used to calculate IOP manually, these operations are preferably computerized. Accordingly, the instant tonometer preferably includes a data acquisition unit programmed to control tonometer operation, data acquisition and processing of the data.

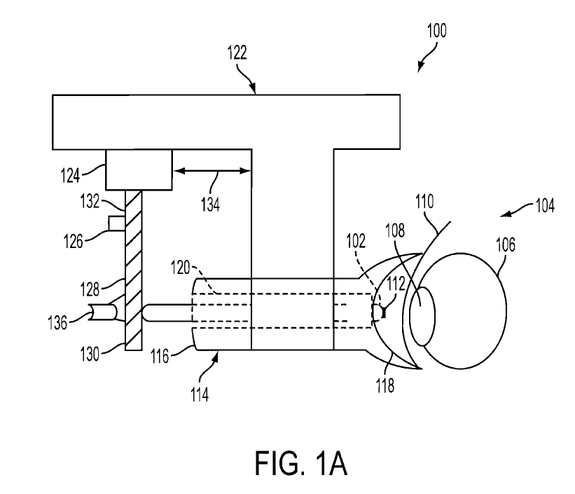

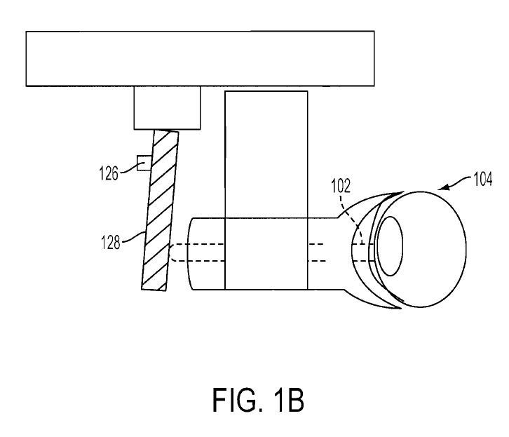

FIG. 1 depicts an exemplary tonometer 100 comprising an ocular probe 102 to be pressed into contact with a patient's eye 104. The eye generally comprises an eyeball or a globe 106, a cornea 108 and an eyelid 110. The ocular probe 102 may be hollow or solid and may be made from glass or plastic. The cross-section of the ocular probe 102 may be square, circular, or any other shape suitable for exerting force on a patient's cornea. At least a part of the distal tip 112 of the ocular probe 102 is preferably flat and has a known area A. In one embodiment, the ocular probe is a circular rod of between about 2.8 mm to about 3.8 mm, with about 3.3 mm being preferred. Accordingly, the cross-sectional area of the probe that comes in contact with the eyelid is between about 6.1 and about 11.3 mm², and more preferably between about 6.5 mm² and 10.5 mm², with 8.5 mm² being preferred.

To ensure that the eyeball does not move during the test, the instant tonometer may include an ocular stabilizer 114. In some embodiments, the ocular stabilizer may comprise a cylinder 116 and a cup 118. The cylinder is held by a frame 122 and defines a channel 120 in which the ocular probe moves relative to the frame 122 and the ocular stabilizer 114. The cup is crescent-shaped and is sized to form a snug fit around the eyeball over the eyelid to reduce or preferably eliminate movement of the eye during the test, including involuntary movement. In some embodiments, the cup has a diameter of 7 to 17 mm, with 12 mm being preferred. Another benefit of using the ocular stabilizer is that it also ensures that the eyelid also stays stationary during the test so not to skew the measurements.

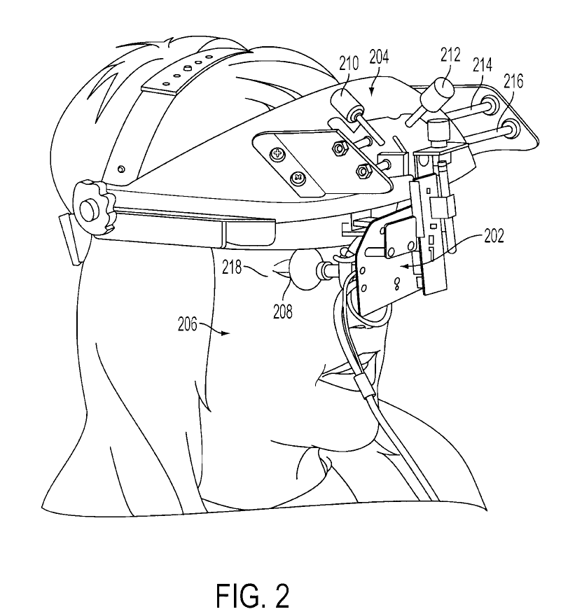

Additionally or alternatively, the tonometer itself may also be stabilized to further improve reproducibility and accuracy of the measurements. To that end, the frame can be mounted to the head of the patient or a desk. Referring to FIG. 2, the instant tonometer 202 is secured to a head-mount 204 to be worn by the patient 206 while the test is being performed. As can be seen from FIG. 2, the tonometer 202 can swing in and out of position or be moved sideways as necessary along the rails 214, 216 to place the ocular stabilizer 208 and ocular probe (not shown) in contact with the patient's eye to be tested 218, which may be referred herein as the test eye. Once the tonometer is in the proper position, it can be secured in that position using set screws 210, 212.

Referring back to FIG. 1, in operation, the ocular probe 102 is moved by a motor 124 along a linear path 134. This causes the ocular probe 102 to apply force F to the eye 104 as shown in FIG. 1b. For safety reasons, it is preferable not to apply force exceeding 3.0 grams. In one embodiment, a translation motor 124 may be utilized for moving the ocular probe 102. One example of a suitable motor is JR Sport Servo MC35 micro marketed by Horizon Hobby, Inc., Champaign, Ill. Other feather, micro or mini sized servo motors may be used.

The motor may be activated by the user pressing a mechanical on/off switch or by a proximity switch that is activated when the user places the tonometer in proximity of the test eye, as known in the art.

The position of the ocular probe at a given time is monitored by a position monitor. Such position monitor may be a part of the translation motor or a separate unit. Monitoring the ocular probe's position may be accomplished by using a variety of devices such as, for example, an ultrasound transducer, a laser range finder, or a linear variable displacement transducer (LVDT), with an LVDT being preferred. Generally, an LVDT produces an alternating current output voltage that is proportional to the mechanical displacement of a small iron core. Here, the core of the LVDT may be linked directly or indirectly to the ocular probe, and the position of the ocular probe may be determined from the voltage signal from the LVDT.

The force exerted by the ocular probe 102 on the eye 104 is measured by a force sensor 126. In some embodiments, the force sensor 126 includes a piezo-resistive element in the form of a Wheatstone bridge so that it balances out thermal variations. Suitable force sensors are preferably capable of measuring small force in the range of between about 0 gm to about 5 gm, and a force difference of approximately 10⁻² gm.

The force sensor 126 may be coupled to the ocular probe either directly or indirectly. As shown in FIG. 1, in one embodiment, the sensor 126 is coupled to the ocular probe 102 through a spring 128. One end 132 of the spring 128 is in contact with the force sensor 126 whereas the other end 130 is in contact with the ocular probe 102. Ideally, the spring 128 is selected to have an effective compressibility similar to the eye, to maximize the sensitivity of the device and minimize the amount of force needed to take a measurement. Referring to FIG. 1a, when the ocular probe is pushed against the eye, the force (F) on the probe 102 compresses the spring 128 and the compressive force on the spring is measured by the force sensor 124. One example of a suitable spring is a steel blade spring a with a spring constant comparable to that of the eye, which is determined by the thickness of the spring.

The inaccuracies in positioning of the ocular probe on the center of the cornea may lead to inaccurate results. Similar issues may also arise from motion of the eye during the test. Accordingly, the instant tonometer also includes a mechanism for aligning the ocular probe with the center of the cornea through the eyelid and, in some embodiments, an ocular stabilizer, as described above.

Referring back to FIG. 1, in one embodiment of the instant tonometer the mechanism for aligning the ocular probe includes a light source 136 mounted behind the ocular probe, i.e., at the end of the ocular probe 102 opposite the end that comes in contact with the eye, along the centerline of the ocular probe 102. In such embodiment, the ocular probe may be transparent so the patient can clearly see the light from the light source 136 and fixate his or her eye on it. The light source may include a light emitting diode (LED), a laser, or any other type of light source.

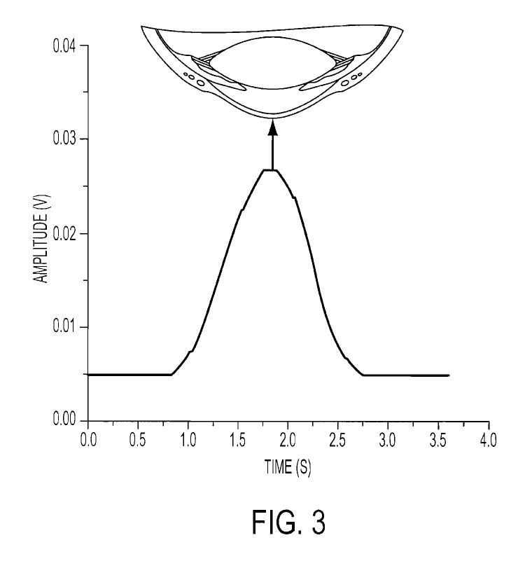

In another embodiment, the mechanism for aligning the ocular probe utilizes the position monitor and/or force sensor to center the probe on the cornea. While the probe is placed on the eyelid of the closed eye, the open eye is moved around thereby causing the closed eye to move as well. Because the cornea protrudes beyond the spherical radius of the eyeball and the center of the cornea protrudes the most, the signal from the force sensor and/or the displacement monitor will peak when the ocular probe is precisely at the center of the cornea, as shown in FIG. 3. The patient is notified that the ocular probe is centered by, for example, an audible signal. Hearing the signal prompts the patient to hold still and start the test.

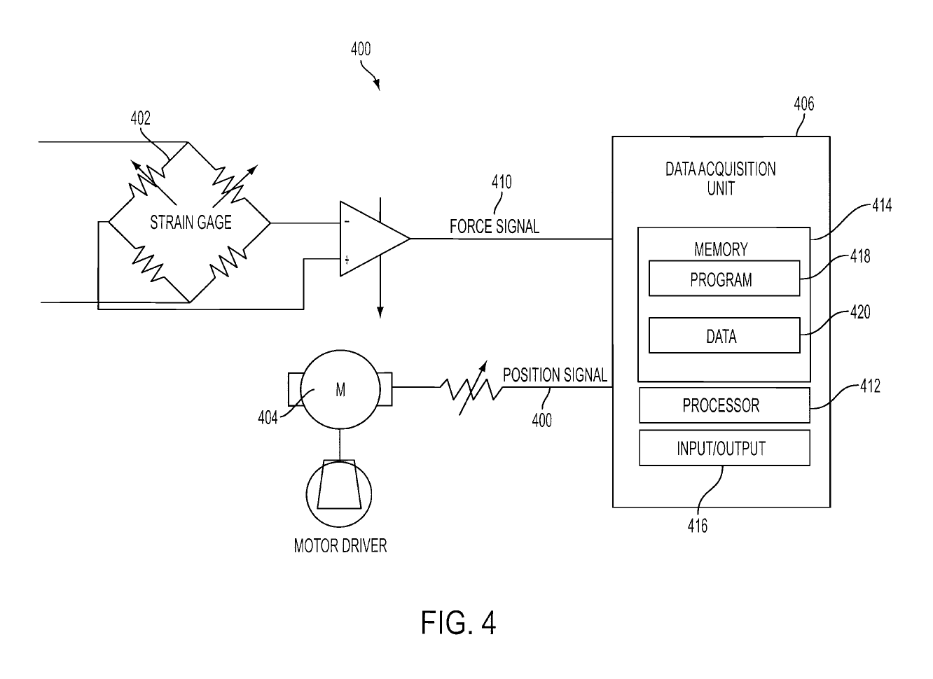

As noted above, although the data from the force sensor and position monitor may be collected and/or used to calculate IOP manually, these operations are preferably computerized. An exemplary schematic diagram of the electronics 400 for operating the instant tonometer is presented in FIG. 4. Both the force sensor 402 and the position monitor 404 are in communication with a data acquisition unit 406. The signals 408, 410 from the position monitor 404 and the force sensor 402, respectively, to the data acquisition unit 406 may be conditioned, i.e. amplified and/or filtered, as necessary.

A suitable data acquisition unit 406 includes at least one processor 412, in communications with memory 414 and input/output (I/O) circuitry 416. In some embodiments, the I/O circuitry 416 may be integral with the processor 412. The memory 414 includes program code 418 and data 420. The program code 418 is executable by the processor 412 and is used to control the operations of the tonometer and process the data, as applicable. The data 420 may include any data needed by the program code 418 to effect the desired operations.

The I/O circuitry 416 is used to facilitate communications between the processor 412 and force sensor and position monitor, as known in the art. For example, the data acquisition unit 400 may collect data from the force sensor 402 and position monitor 404 according to a predetermined collection routine and store sample data in the memory 414. In some embodiments, the duration of one measurement is approximately 2 second, and up to 12 measurements per minute can be taken. The data may then be retrieved for analysis, such as by the data acquisition unit itself or by downloading to an external computer, as known in the art.

To facilitate the mechanism for centering the probe on the cornea, the memory of the data acquisition unit includes a program code to accomplish one or more of the following: to cause the processor to receive data from the position monitor and/or the force sensor; to detect placement of the tonometer's ocular probe against a patient's eyelid, which may be inferred from the signal from the force sensor and/or position monitor; to identify when the ocular probe is centered against the patient's cornea as inferred from the signal from the force sensor and/or position monitor; to and notify the user that the ocular probe is centered against the cornea and the test can commence.

The data acquisition unit is also used to determine a value indicative of IOP. The term “value indicative of IOP” as used herein means an absolute value of IOP as well as any value having a known or ascertainable relationship to IOP and thus indirectly indicating the value of IOP. To this end, the program code is executable by the processor to also cause the processor to receive data from the position monitor and the force sensor and to determine from this data a value indicative of IOP.

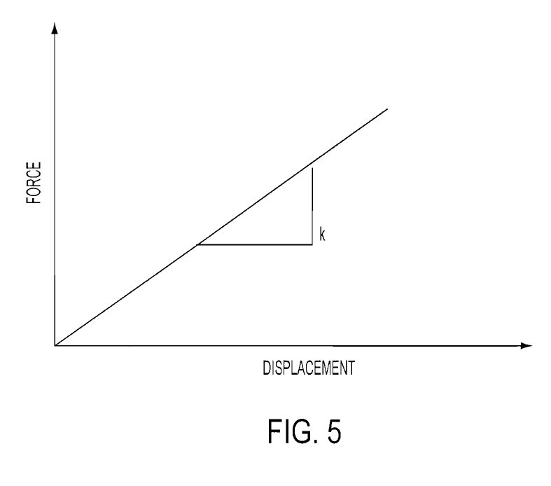

The value indicative of IOP can be calculated as following. Because the eyelid and the cornea have a linear compressibility, a typical graph of the force on the probe as a function of the displacement of the probe is shown in FIG. 5. The relationship between the force and displacement can be approximated as F=k*X, where F is the force, X is the displacement, and k is the combined elastic constant or the inverse of compressibility of the eyelid and the cornea. In other words, k is the slope of a linear fit to the measurements of force on the probe as a function of distance that a probe has moved after touching the eyelid. The point when the probe first touches the eyelid is referred to herein as initial contact point. The initial contact point can be determined from the force measurements. Accordingly, k can easily be calculated from the data obtained from the position monitor and the force sensor. The value of k is indicative of IOP, with larger value of k indicating higher IOP. Accordingly, in some embodiments variations in IOP may be monitored by observing the value of k.

Additionally or alternatively, this data can further be used to calculate a value of IOP as follows. Since the tests using the instant tonometer are performed through the eyelid, the overall compressibility of the system can be approximated as the sum of the compressibility of the eyelid and compressibility of the cornea. This relationship can be expressed as 1/k=1/k_{cornea}+₁/k_{eyelid}, wherein 1/k_{cornea} is the compressibility of the cornea and 1/k_{eyelid} is the compressibility of the eyelid. It should be noted that compressibility of the retropulsive structure (1/kᵣₚₛ) also contributes to the overall compressibility, but because it is much smaller than the compressibility of the cornea and the compressibility of the eyelid it can typically be ignored.

K_{eyelid} can be obtained for a particular patient in advance by, for example, a combination of the test data using the instant tonometer with an aplantation measurement touching the cornea. Other methods for determining the value of k_{eyelid} are described below in the Examples. Having determined the values of k_{eyelid} and k, k_{cornea} can easily be calculated from the equation above.

Once k_{cornea} has been calculated, it can be used to approximate IOP at a known distance into the eye using the following formula: IOP=k_{cornea}*x₀)/A, wherein A is the area of the distal tip of the ocular probe, which is known, and x₀ is the displacement of the probe. The displacement of the probe x₀ is a constant standard distance moved toward the eye from the initial contact point, with such distance being preferably between about 0.1 mm and about 0.2 mm. A more precise value of IOP may be calculated using the following formula:

IOP=(k_{cornea}*x₀)/A)*0.736.

In operation of the preferred embodiment, the patient or a care-giver positions the head-mount on the patient's head and positions the tonometer so the ocular probe gently touches the closed eyelid of the test eye, i.e. the eye to be tested. The mechanism for aligning the ocular probe with the center of the cornea is activated to center the probe. The patient moves the open eye left, right, up or down so that the test eye follows, until a peak signal from the force sensor and/or position monitor is received by the data acquisition unit, which immediately notifies the patient with an audible signal that the ocular probe is centered on the cornea.

While holding still, the patient secures the ocular stabilizer in place and activates the motor, which pushes the ocular probe against the eye. The force on the probe and the probe's distance into the eye are measured by the force sensor and position monitor, respectively, and are communicated to the data acquisition unit. The data acquisition unit uses these data to calculate a value representative of IOP and notifies the patient when the test is completed.

### EXAMPLES

### Example I

### Ability to Correct for Errors in IOP Measurements Using Various Tonometers

In the measurement of IOP using different methods of tonometry, studies have shown that many factors influence the value of IOP measured. Some factors are: corneal thickness (See Sandhu et al., J. Glaucoma, 14, 215-218 (2005)), rigidity of the ocular coat and elasticity of the eyeball (which includes the compressibility of the intraocular vascular bed). (See Friedenwald et al., “Modern refinements in tonometry,” Documenta Opthalmologica, 4, 335-362 (1950) and Friedenwald, Am. J. Opthalmol., 20, 985-1024 (1937)).

Friedenwald in his work used the results of previous work on the rigidity, elasticity of the eye and distensibility of the eyeball and the IOP measurements obtained using the Schiotz nomogram to determine the resistance of the ocular coat to deformation so as to use this value to correct for pressure readings obtained using the Schiotz Tonometer. He also noted that variations in the elasticity of the cornea have the same effect on the tonometer readings as variations in the elasticity of the eye as a whole. (See Friedenwald et al., “Modern refinements in tonometry” and Whitacre et al., Survey Ophthalm., 38, 1-30 (1993).)

Friedenwald derived a mathematical relationship between the pressure in the eye before and during tonometry, the ocular rigidity and the volume of fluid displaced. He also used a similar relationship to calculate the correction to be applied to the value of IOP measured using the Schiotz Tonometer. Friedenwald noted that patients with deep physiological cupping of the optic disc tended to show rather low values in their rigidity coefficient. (See Friedenwald et al., “Modern refinements in tonometry”). No numerical value has been assigned to the size and depth of this physiological cup yet.

In accounting for the sources of errors in the measurement of IOP, error resulting from the compressibility of the structural support of the eye, which is referred to herein as the posterior retropulsive structure (RPS), is typically neglected. This error could be negligible in people with a high coefficient of compressibility but not for those with a lower value. Low compressibility may give a much lower underestimation of the measured IOP.

In the measurement of the IOP using the transpalpebral tonometer of the present invention, which exploits the compressibility of the eyelid and the ocular medium to determine the value of the IOP, means of correcting for the effect of compressibility of the retropulsive structure have been devised. It is assumed that the eyelid, cornea and its content, called the ocular media, and the retropulsive structure are each compressible elastic media, the effective compressibility of which is the sum of all three given as (β=β_{lid}+β_{cornea}+βᵣₑₜᵣₒ). Compressibility is given as β=−(1/V)*(δV/δP), where V is the volume of the indented region and P is the pressure. The probe may have a constant area with a diameter of 3.06 mm. The compressibility of the combined ocular media can be expressed as (A/x)*(δx/δF), where x₀ is a characteristic distance of aplanation, calculated to be 0.15 mm, F is the force applied through a given distance x, and (δx/δF) is the inverse slope of the plot of force as a function of distance.

### Example II

### Model for IOP Measurement

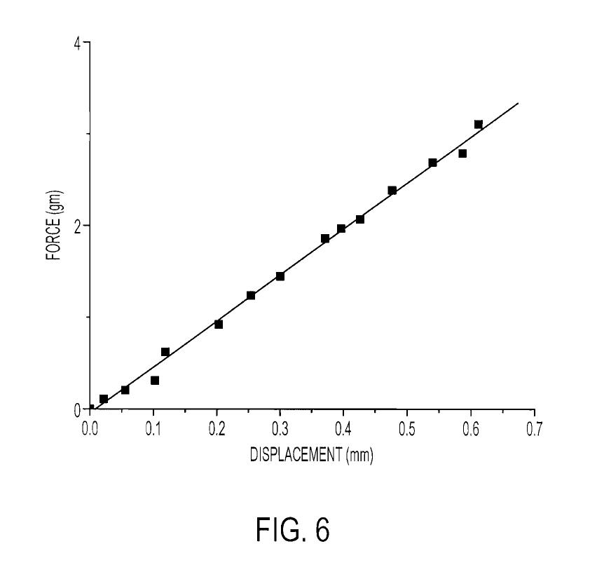

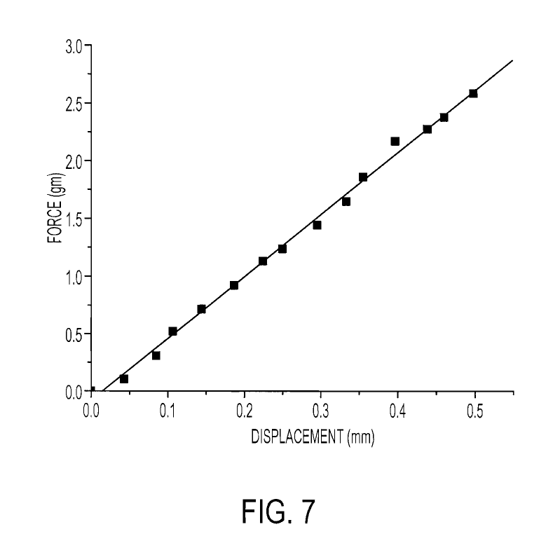

To determine the coefficient of the compressibility of the posterior retropulsive structure using the tonometer of the present invention, a graph was plotted of force as a function of the distance used to compress the RPS by placing a shield over the cornea. The graph is shown in FIG. 6, in which the inverse of the slope is the combined compressibility of the shield and the RPS. Another graph was prepared of force as a function of distance for a very hard structure such as a wall, as shown in FIG. 7, to determine the compressibility of the spring of the instrument. The difference between the compressibility of the instrument and the combined compressibility of the cornea shield and the RPS gives the compressibility of the RPS, as follows:

$$\frac{1}{{k}_{rps}} = \frac{{k}_{shield + RPS} * {k}_{wall}}{{k}_{shield + RPS} - {k}_{wall}}$$

This measurement was done on three human subjects and it was found that kᵣₚₛ and thus its inverse (1/kᵣₚₛ) is different for each patient, with a mean value of 1/kᵣₚₛ of about 0.021 mm/gmf.

Once the retropulsive structures are characterized, the information can be used to measure the IOP of a subject. First, the assumption was tested that, for the composite ocular structure, the relation between force, f, and displacement, x, is linear and given by: f=kx, where k is the rigidity or the inverse of the compressibility, with k independent of x. The parameter, k, if proven to be a constant, corresponds to the slope of a linear fit to the measurements of force as a function of distance. For composite media, the combined compressibility is given as:

$$\frac{1}{k} = \frac{1}{{k}_{eyelid}} + \frac{1}{{k}_{cornea}} + \frac{1}{{k}_{RPS}}$$

These media are in series when the eye is closed. The elastic constant is the inverse of the compressibility of the media. Knowing the compressibility of the composite media, it is feasible to determine the ocular compressibility when the compressibility of the eyelid, which can be determined separately, is known. The great body of evidence from studies of Goldman and other tonometers indicates that the ocular compressibility is directly related to the intra-ocular pressure. (See e.g., Harrington et al., Arch. Ophthal., 26, 859-885 (1941) and Friedenwald et al. “Modern refinements in tonometry.”)

The inverse of slope of the linear regression of the graph of force as a function of distance is the combined compressibility of the eyelid and the ocular medium. The compressibility of the eyelid is determined separately by a similar method. A corneal shield is placed over the eye, the probe is aligned at the center of the shield, and the force as a function of distance is recorded. The inverse of slope of a linear fit to the data gives the compressibility of the retropulsive structure k_{shield}. The eyelid is closed over the cornea shield and the probe is aligned to be at the center of the upper eyelid. The force as a function of distance is again recorded, the inverse of slope of the least squares fit to the data, gives the combined compressibility of the retropulsive structure and the eyelid, k_{lid+shield}. Since the compressibility of the retropulsive structure alone has been determined, we can determine the compressibility of the eyelid only, using this expression:

$$\frac{1}{{k}_{lid}} = \frac{{k}_{lid + shield} * {k}_{shield}}{{k}_{lid + shield} - {k}_{shield}}$$

Using the compressibility of the eyelid, the ocular compressible is calculated from the combined slope of the eyelid and the ocular medium as:

$$\frac{1}{{k}_{cornea}} = \frac{{k}_{lid + cornea} * {k}_{lid}}{{k}_{lid + cornea} - {k}_{lid}}$$

This compressibility can then be converted to pressure as follows:

$$P ( mmHg ) = \frac{{k}_{cornea} * x}{A} * 0.736 ,$$

where k_{cornea} is the inverse of compressibility of cornea, x is the displacement of the probe, preferably a constant standard distance moved toward the eye (similar to the distance in Goldmann aplanation tonometry), and A is the are of the ocular probe.

### Example III

### Testing of Model for IOP Measurements Using the Instant Tonometer

The transpalpebral tonometer of the present invention was tested and it was found that the measurements obtained were in agreement with the fundamental assumptions made about the nature of the media of interest, that is, the eyelid and the ocular medium (the cornea and its contents) within the 3.30 mm diameter probing region. Of the measurements that were made, 50% have a standard deviation less than or equal to 0.050 from linear. This degree of deviation from linear is statistically acceptable.

The reason why the other 50% have a higher standard deviation came from either the unconscious twitching of the eyelid or movement of the subjects during measurements. Sometimes the subjects lean off from the chin-rest, causing the probe to touch a very small insignificant part of the eye. (The travel distance of the motor is fixed, 1.5 mm). The same reason can be used to explain why the same 50% of the measurements have a mean pressure difference ΔP between measurements to be more than 2.5 mm Hg, which is the acceptable pressure difference for commercial devices. (See e.g. Resua, et al., Optometry and Vision Sci., 82, 143-150 (2005) and Sacca et al., Opthalmol., 212, 115-119 (1998).)

Another explanation for 50% of the measurements having a mean pressure difference greater than 2.5 mm Hg could be that, if the rigidity of the eyelid is constant as assumed, the probing might be slightly off the center of the cornea at different times, because it has been proven that different parts of the cornea have different rigidity (See Cheng et al., Clin. Exper. Opthalmol., 33, 153-157 (2005)). This was corrected by using a cup of a 12 mm diameter that helds the entire cornea in place during measurements, so as to reduce involuntary motion of the eye.

Another possible source of the error could be the slight displacement of ocular fluid, during each repeated probing.

However, this error is not subject to significant control and, regardless, it has been shown that repeated tonometery gives slightly different values of IOP measured. (See Sandhu et al., J. Glaucoma, 14, 215-218, 2005.)

### Example IV

### Tonometer Functionality

The functionality of the device of the present invention, including its sensitivity, reproducibility and linear behavior, was tested both in the lab and on human subjects. The device can measure a force difference of about 0.01 gm (This force value with a probe tip diameter of 3.30 mm corresponds to a pressure value of 0.085 mm Hg) within a distance of 0.01 mm.

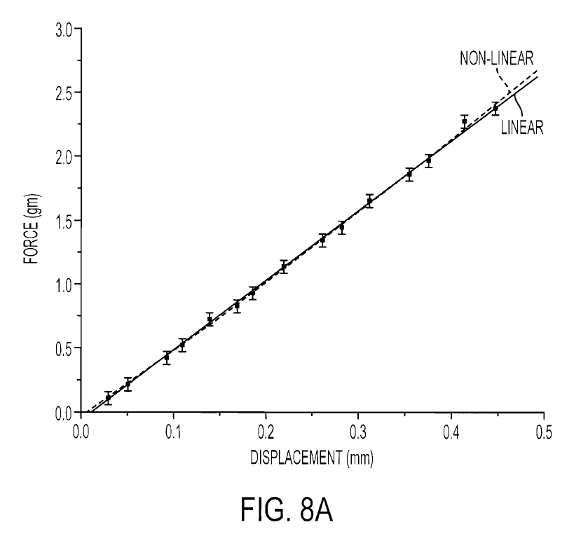

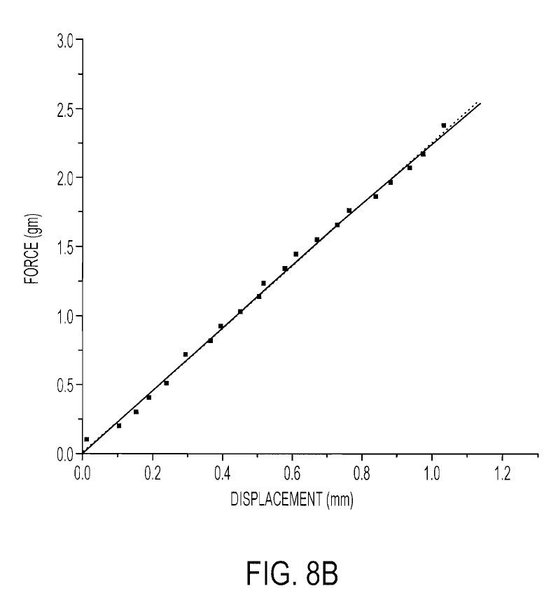

The graph in FIG. 8a shows the testing of the sensitivity and the linear performance of the device in the lab. The error bars on the graph are only 0.5%. The data shows a force sensitivity of less than 0.05 gm, a displace-ment sensitivity of less than 0.02 mm, and a compressibility standard deviation of 0.034. The sensitivity indicates that the tonometer can make measurements within a wide range of displacements of the eye, using very gentle probes. The small uncertainty indicates that the device is capable of high accuracy.

The linear behavior of the device was tested by comparing a linear fit and quadratic fit on the same data set taken in the lab. The standard deviation from the linear fit was about 0.034 and the standard deviation from the quadratic fit was about 0.030. The coefficient of regression (r) from the linear fit was about 0.99909 and that from the quadratic fit was about 0.9987. There was some element of non-linearity in the device which could be the behavior of the metal on which the bridge is mounted. Such nonlinearity may be corrected by mounting the bridge on a material that has a linear behavior. Comparison of a non-linear fit to this data shows that the next higher-order term above linear is smaller by a factor of 100 at x=1 mm and that the standard deviation in the 2^{nd} order fit is comparable to the 1st order fit within the uncertainty.

The graph in FIG. 8b shows the testing of the sensitivity and the linear property of the device on human subjects. The results are very similar to the ones obtained in laboratory tests. The standard deviation from the linear fit (solid line) was about 0.035 and the standard deviation from the quadratic fit (broken line) was about 0.0389. The coefficient of regression (r) from the linear fit was about 0.9986 and that from the quadratic fit was about 0.9973. The results show that the best fit to the data is a linear fit, which allows you to calculate the slope and in turn, a value indicative of IOP, including an actual value of IOP.

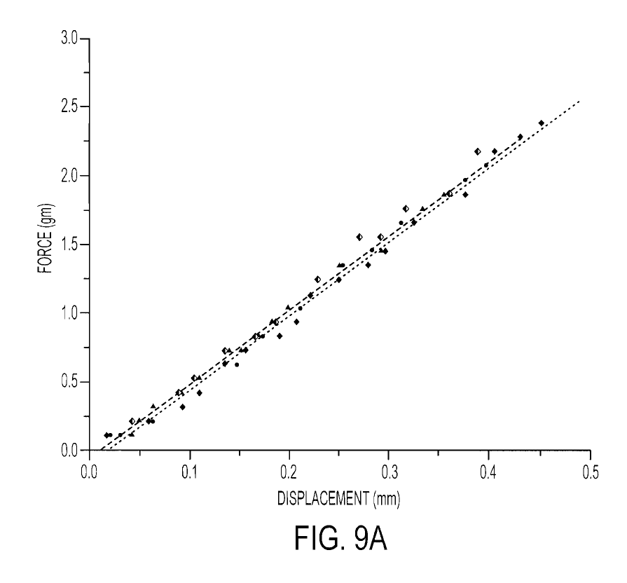

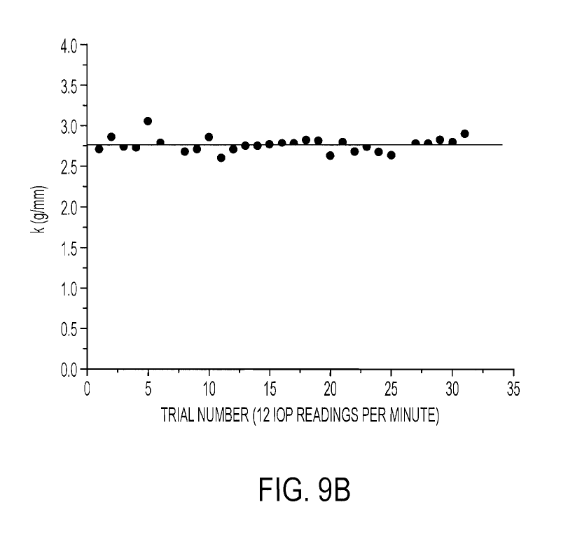

The measurement reproducibility for the device of the present invention was tested and the coefficient of variation (CV), which is a measure of its reproducibility, was calculated to be only 1.7%, which is about five times less than the CV for Goldmann applanation tonometers. Referring to FIG. 9a, the mean was 5.2 and the standard deviation was 0.09. The measurement accuracy of devices according to the present invention are about +/−2 mm Hg of the IOP. The data on the reproducibility of the results obtained with the tonometer of the present invention is presented in FIG. 9b. With proper alignment, the reproducibility of the results will be to a standard deviation within 0.5 mm Hg for a patient with an average IOP of 16 mm Hg.

### Example V

### Reproducibility of Test Results

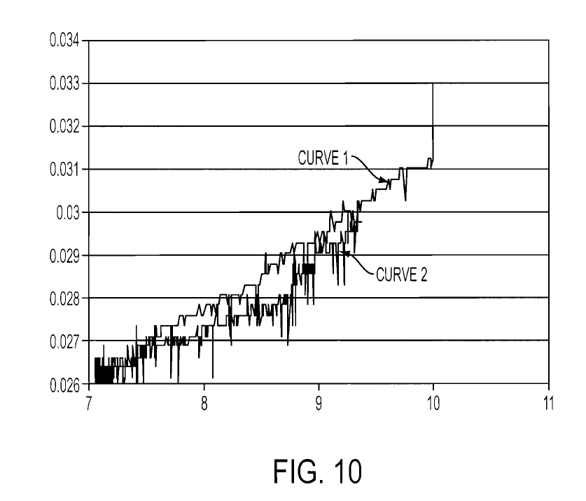

In FIG. 10, the upper curve (curve 1) presents data taken while the ocular probe was held relatively steadily while the lower curve (curve 2) presents data taken while the ocular probe was less stable. As can be clearly seen from this example, stabilizing the tonometer results in more consistent measurements.

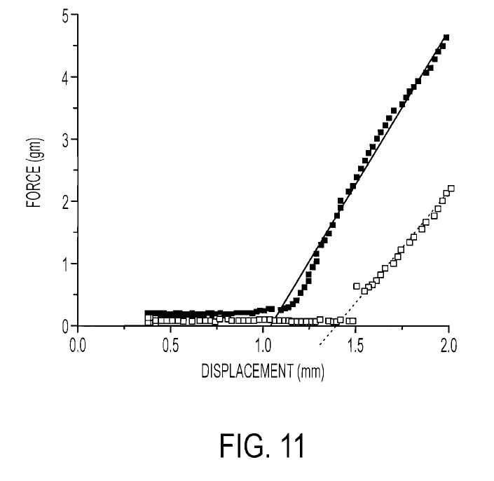

FIG. 11 depicts two curves that represent data from tests on the same eye at about the same time. Nonetheless, these curves are not consistent which was determined to be caused by variations in the position of the ocular probe on the center of cornea. The equation for the upper curve was calculated to be y=4.96479*x−5.8544, whereas the equation for the lower curve was calculated to be y=3.54204*x−4.937.

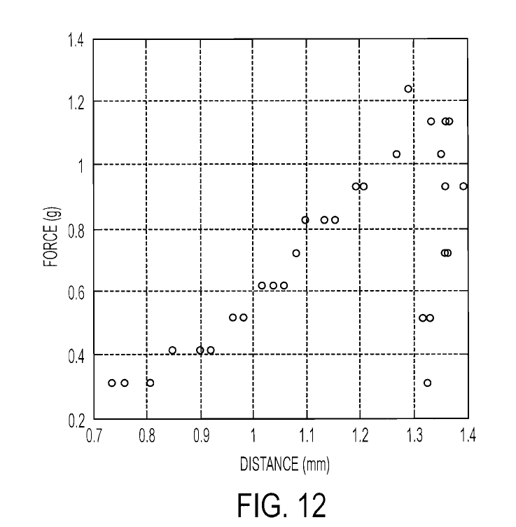

FIG. 12 presents a graph showing variations in force as a function of the ocular probe moving toward the eye for a position of the ocular probe that is off the corneal center, but still on the cornea. As can be seen from these figures, the position of the probe is a major contributor to the variations in the data.

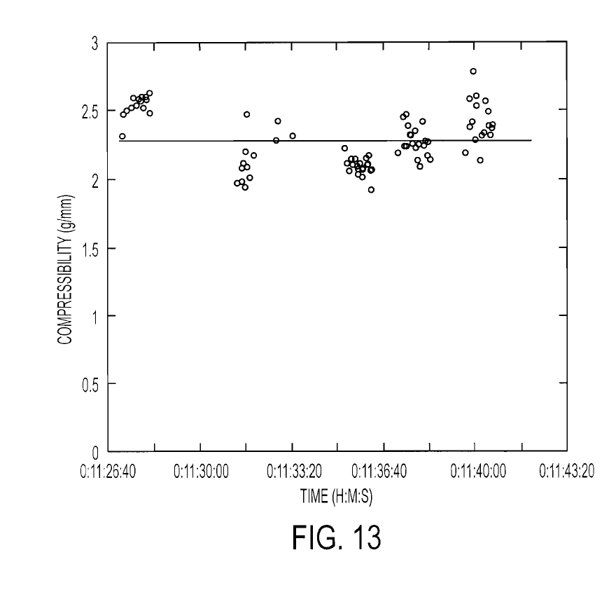

Next, the accuracy of the tonometer of the present invention was measured in experiments in which the patient takes the device off between measurement sessions and therefore must reposition it. This protocol assesses the positioning accuracy. The results are shown in FIG. 13. There is lower accuracy in this case, indicating that lack of patient care can influence results. The results (30 measurements to determine each point and 12-15 points in each group) show that the variation among groups of measurements is comparable to that within each group. A solid line indicating constant eye pressure is shown for comparison.

### Example VI

### Compliance Map of Cornea Through the Eyelid

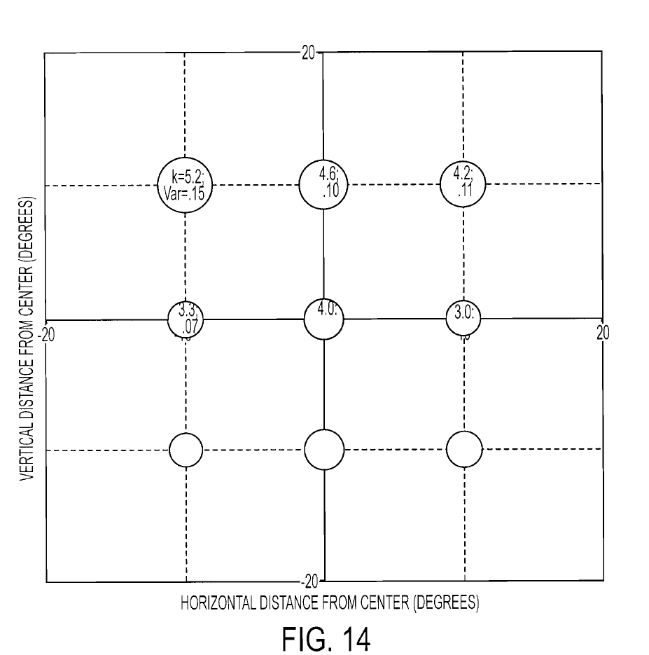

The variation in readings resulting from variations in the placement location of the probe above the eye was documented and the results are presented in FIG. 14. The patient kept the tonometer in the same place on the head and then looked at a series of points on a wall grid depicted in the figure. The magnitude of the measurement compliance is depicted by the size of the solid circles. These results confirm the hypothesis that the positioning of the eye, i.e, centering the probe on the cornea, contributes substantially to measurement accuracy.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention which is defined by the following claims.

What is claimed is:

1. A tonometer for measurement of intraocular pressure comprising:
an ocular probe movable in a linear manner by a motor against the closed eyelid of a user's eye to be tested;
a distance sensor configured to monitor the probe's position as the probe is moved against the eyelid and provide a distance measurement;
an mechanism for aligning the probe with the center of the cornea underneath said closed eyelid; and
a force sensor configured to measure force on the ocular probe as it is moved against said closed eyelid by said motor at said cornea center and provide a force measurement;
wherein a value indicative of the intraocular pressure (IOP) of the test eye is determined from said force and distance measurements.

2. The tonometer of claim 1, wherein the probe is moved a predetermined distance between about 0.1 mm and about 0.2 mm.

3. The tonometer of claim 1 further comprising a frame, where the ocular probe is stabilized by said frame and is movable in relation to the frame.

4. The tonometer of claim 3, wherein the frame comprises a head-mount.

5. The tonometer of claim 3, wherein the frame comprises a desk-mount.

6. The tonometer of claim 1 further comprising a data acquisition unit comprising a processor and memory coupled to said processor, said memory comprising a program code executable by said processor to cause said processor to receive data from the distance sensor and the force sensor and calculate IOP in said test eye from said data.

7. The tonometer of claim 1 further comprising an ocular stabilizer shaped to be placed around an eyeball and positioned to keep the eyeball of said test eye from moving while said measurements are being taken.

8. The tonometer of claim 1, wherein said ocular probe is transparent, and said mechanism for aligning the ocular probe with the center of the cornea underneath said closed eyelid comprises a light source positioned behind the probe in alignment with the center of the probe.

9. The tonometer of claim 6, wherein the memory of the data acquisition unit further comprises a program code executable by the processor to cause the processor to perform the following steps: detecting placement of the ocular probe against a user's eyelid; identifying when the user's cornea is centered against the ocular probe, and notifying the user that the eyeball is centered.

10. The device of claim 9, wherein said adjustment mechanism comprises an audible feedback mechanism that signals the user when the center of said ocular probe is aligned with said cornea center.

11. The of claim 10, wherein the program code to execute the step of identifying when the eye is centered comprises the program code to execute the step of detecting the maximum force on the probe by the eyeball.

12. The tonometer of claim 10 further comprising a spring adapted to keep the probe against the eyelid.

13. The tonometer of claim 1 wherein the ocular probe is connected to the force sensor with a spring having substantially the same compressibility as the eye.

14. A method for the measurement of intraocular pressure in a test eye of a patient, the method comprising:
placing a tonometer having an ocular probe in proximity to the test eye of said patient, said tonometer comprising an adjustment mechanism for aligning the probe with the center of the cornea of the test eye through a closed eyelid, and emitting a signal when the probe is aligned with the cornea center;
closing the eyelid of said test eye and placing the ocular probe in contact with said closed eyelid;
aligning the probe with the center of the cornea by moving the observing eye opposite said test eye in at least one direction selected from the group consisting of left, right, up and down, so that said closed eye follows, until a signal is received that the ocular probe is aligned with said cornea center;
causing advancement of the probe a predetermined distance against the closed eyelid above the cornea center while measuring the force required to deflect the eyelid and cornea as a function of distance that the probe has moved after touching the eyelid; and
calculating a value indicative of IOP from said force measurement.

15. The method of claim 14, wherein the step of aligning the probe with the center of the cornea comprises the steps of measuring the distance between the probe and the cornea through the closed eyelid, and identifying the cornea center by its protrusion from the eyeball.

16. The method of claim 14, wherein the representative value of IOP is the actual value of IOP, which is calculated by:
recording a series of force readings on the ocular probe as a function of its displacement against a closed eyelid;
calculating the overall coefficient of compressibil-ity from the formula F=kx, wherein F is the force on the probe, x is the displacement of the probe, and k is the inverse of the overall coefficient of compressibility;
calculating the coefficient of compressibility of the eye from the formula 1/k=1/k_{eye}+1/k_{eydlid}, wherein 1/k_{cornea} is the coefficient of compressibility of the eye and 1/k_{eydlid} is the coefficient of compressibility of the eyelid; and
calculating IOP from the formula:
P=(k_{cornea}*x₀)/A,
wherein P is IOP, A is the cross-sectional area of the front end of the probe, and x₀ is initial contact point.

17. The method of claim 14, wherein the representative value of IOP is the overall coefficient of compressibility, which is calculated by:
recording a series of force readings on the eye as a function of displacement of the probe; and
calculating the overall coefficient of compressibil-ity from the formula F=kx, wherein F is a force on the probe, x is displacement of the probe, and k is the inverse of the overall coefficient of compressibility.
